(19) United States
(12) Patent Application Publication
(10) Pub. No.: US 2026/0007604 A1
(43) Pub. Date: Jan. 8, 2026

(54) USE OF A SPRAY FREEZE-DRYING PROCESS FOR THE LYOPHILIZATION OF A mRNA-ENCAPSULATING LIPID NANOPARTICLES FORMULATION

(71) Applicant: Pfizer Inc., New York, NY (US)

(72) Inventors: Bakul Subodh Bhatnagar, North Reading, MA (US); Ramin Darvari, Lexington, MA (US); Israel Borges Sebastiao, Lowell, MA (US); Serguei Tchessalov, North Billerica, MA (US)

(73) Assignee: Pfizer Inc., New York, NY (US)

(21) Appl. No.: 18/875,885

(22) PCT Filed: Jun. 20, 2023
(86) PCT No.: PCT/IB2023/056374

§ 371 Date: Dec. 17, 2024

Related U.S. Application Data
(60) Provisional application No. 63/354,835, filed on Jun. 23, 2022.
(60) Provisional application No. 63/382,895, filed on Nov. 9, 2022.

(51) Int. Cl. A61K 9/16; A61K 9/127; A61K 9/19; A61K 9/51; A61K 31/7105; B82Y 5/00

(52) U.S. Cl. CPC A61K 9/1682; A61K 9/127; A61K 9/1623; A61K 9/19; A61K 9/5123; A61K 31/7105; B82Y 5/00

(57) ABSTRACT

The invention relates to the lyophilization of a liquid pharmaceutical formulation including lipid nanoparticles encapsulating mRNA.

According to the invention, a spray freeze-drying process is used to achieve the lyophilization.

The invention is of particular interest for the lyophilization of mRNA vaccine formulations.

### TECHNICAL FIELD

The present invention relates to the lyophilization of liquid pharmaceutical formulations that include lipid nanoparticles encapsulating messenger RNA.

### BACKGROUND OF THE INVENTION

In the pharmaceutical field, an increasing number of promising gene therapies and vaccines are based on RNA and DNA polymers. A critical issue associated with the implementation of such RNA- or DNA-based gene therapies or vaccines is delivery. Naked RNA or DNA molecules are rapidly degraded in biological fluids, do not accumulate in tissues following systemic administration, and cannot penetrate target cells, even if they get to the target tissues. Further, the immune system is designed to recognize and destroy vectors containing genetic information.

It has therefore been proposed to administer RNA or DNA molecules encapsulated in lipid nanoparticles (LNPs) such that the RNA or DNA molecules can be delivered to the target cells without degradation.

In the case of an RNA vaccine, in particular a messenger RNA (mRNA) vaccine, LNPs aid delivery of RNA to cells and thereby promote an immunological response. The formation of the LNPs and the encapsulation of the RNA is critical to the efficacy of the vaccine and the manufacturing operations bringing the RNA and the lipid material together must be done in appropriate conditions to enable encapsulation.

In the context of a pandemic outbreak, a number of challenges are associated with the supply chain of vaccines for allowing rapid access to such vaccines to the greatest number of people around the globe.

A first major challenge is related to the required storage temperature for vaccines, such as mRNA vaccines, that require colder (≤−20° C.) storage. Freeze-drying technologies can mitigate the supply chain challenges associated with the storage temperature, as lyophilized products typically only require 2-8° C. storage.

A freeze-drying technology suitable for lyophilizing mRNA vaccines is required to preserve the product attributes and, in particular, to achieve long-term stability of the LNPs, and therefore the performance of the available freeze-drying technologies should be carefully considered in that respect.

Conventional vial freeze-drying (VFD), which is the gold-standard technology for drying of biologicals, has proved suitable for lyophilizing mRNA vaccines. In this regard, reference can be made e.g. to the following publications:
Hiromi Muramatsu, Kieu Lam, Csaba Bajusz, Dorottya Laczkó, Katalin Karikó, Petra Schreiner, Alan Martin, Peter Lutwyche, James Heyes, Norbert Pardi, Lyophilization provides long-term stability for a lipid nanoparticle-formulated, nucleoside-modified mRNA vaccine, Molecular Therapy, 30(5),2022,1941-1951;
Liangxia Ai, Yafei Li, Li Zhou, Hao Zhang, Wenrong Yao, Jinyu Han, Junmiao Wu, Ruiyue Wang, Weijie Wang, Pan Xu, Zhouwang Li, Chengliang Wei, Haobo Chen, Jianqun Liang, Ming Guo, Zhixiang Huang, Xin Wang, Zhen Zhang, Wenjie Xiang, Bin Lv, Peiqi Peng, Shangfeng Zhang, Xuhao Ji, Zhangyi Li, Huiyi Luo, Jianping Chen, Ke Lan, Yong Hu, Lyophilized mRNA-lipid nanoparticle vaccines with long-term stability and high antigenicity against SARS-CoV-2, bioRxiv, Preprint, 2022;
Emily A. Voigt, Alana Gerhardt, Derek Hanson, Peter Battisti, Sierra Reed, Jasneet Singh, Raodoh Mohamath, Madeleine F. Jennewein, Julie Bakken, Samuel Beaver, Christopher Press, Patrick Soon-Shiong, Christopher J. Paddon, Christopher B. Fox, Corey Casper, A self-amplifying RNA vaccine against COVID-19 with long-term room-temperature stability, bioRxiv, Preprint, 2022.

However, the conventional VFD processes have been found to significantly increase the manufacturing time. More specifically, VFD processes may typically have a cycle time of several days, up to 3-7 days, depending on formulations.

There is therefore a need for alternative freeze-drying technologies to accelerate manufacturing, in order to increase the availability to patients of life-saving biological medicines, such as the above-referred mRNA vaccines.

### SUMMARY OF THE INVENTION

According to a first aspect of the present invention, a spray freeze-drying process is used for the lyophilization of a liquid pharmaceutical formulation including lipid nanoparticles encapsulating mRNA.

The inventors have found that the invention allows for drastically reducing the lyophilization cycle time for such formulations, from several days to 2 hours or less.

The invention is not limited to lipid nanoparticles encapsulating a single mRNA construct, but also applies to lipid nanoparticles encapsulating more than one mRNA constructs, in particular mRNAs of different primary structures.

Spray freeze-drying (SFD) is a technology with potential to supply billions of vaccine doses per year, e.g., during a pandemic outbreak, as it allows for manufacturing bulk lyophilized biologicals faster and more efficiently than conventional vial freeze-drying (VFD). Also, it has been found by the inventors that SFD was suitable to preserve the quality attributes of the product and achieve the stability of the LNPs.

According to a preferred embodiment of the invention, the spray freeze dying process comprises the sequential steps of:
spray freezing the liquid pharmaceutical formulation, in a column with temperature-controlled walls, the temperature of which is controlled by means of a cooling agent maintained at temperatures between −100 and −190° C., so as to obtain frozen pellets;
transferring the frozen pellets to a vacuum drying chamber; and
drying the frozen pellets within the vacuum drying chamber at a pressure no higher than 1000 microbar, the pellets being heated at a controlled temperature within the vacuum drying chamber.

According to another aspect of the invention, it is provided a process of lyophilization of a liquid pharmaceutical formulation including lipid nanoparticles encapsulating mRNA, comprising the sequential steps of:
spray freezing the liquid pharmaceutical formulation, in a column with temperature-controlled walls, the temperature of which is controlled by means of a cooling agent maintained at temperatures between −100 and −190° C., so as to obtain frozen pellets;
transferring the frozen pellets to a vacuum drying chamber; and
drying the frozen pellets within a chamber at a pressure no higher than 1000 microbar, the pellets being heated at a controlled temperature within the chamber.

According to a preferred embodiment of the invention, the process may comprise one or more of the following steps:
the frozen pellets are heated within the vacuum drying chamber by direct contact with a temperature-controlled surface provided within the vacuum drying chamber;
the temperature-controlled surface may be formed by the internal surface of a rotary drum within the vacuum drying chamber;
alternatively, the temperature-controlled surface may be formed by the surface of a static shelf or of a set of static shelves within the vacuum drying chamber;
the temperature of the temperature-controlled surface within the vacuum drying chamber is varied within a range of −70° C. to +60° C., preferably of −45° C. to +50° C.;

According to another preferred embodiment, the process may comprise one or more of the following steps:
the frozen pellets are heated within the vacuum drying chamber by contactless heating;
the frozen pellets are heated within the vacuum drying chamber by electromagnetic radiation, in particular infra-red radiation or radio-frequency, in particular microwave, radiation.

According to an optional feature of the invention, the process may comprise, before drying the frozen pellets, a pre-drying step including heating the pellets to an annealing temperature greater than the glass transition temperature of the freeze concentrate (Tg′), but below the ice melting temperature.

Preferably, the pharmaceutical formulation is a vaccine formulation.

In a further aspect of the invention, it is provided a lyophilized pharmaceutical product obtained by a process according to the invention.

In a further aspect of the invention, it is provided a method of reconstitution of a liquid pharmaceutical formulation, wherein a diluent is added to a lyophilized pharmaceutical product obtained by a process according to the invention, to obtain a formulation having an mRNA concentration less than or equal to the mRNA concentration of the formulation prior to lyophilization.

### DETAILED DESCRIPTION OF THE INVENTION

The following definition will be used in the present description and claims:
the term “freeze concentrate” refers to a phase that forms as a consequence of freezing of a pharmaceutical formulation. During freezing, most of the solvent (typically, water) separates from the solution or dispersed phase to form ice. As freezing proceeds with a decrease in the temperature, the unfrozen phase (containing the dissolved solutes or the dispersed phase) becomes progressively concentrated and is described as the “freeze concentrate”. References: (i) Tang X, Pikal M J 2004. Design of Freeze-Drying Processes for Pharmaceuticals: Practical Advice. Pharm Res 21(2):191-200; (ii) Bhatnagar B, Tchessalov S., Lewis L, and Johnson R, Freeze-drying of Biologics. In: Encyclopedia of Pharmaceutical Science and Technology, 4th edition, Publisher Taylor & Francis, 2013:1673-1722.
the term “glass transition temperature of the freeze concentrate” (Tg′), for a certain pharmaceutical formulation, represents the temperature where maximum freeze concentration occurs during freezing (B. Bhatnagar, S. Tchessalov, L. Lewis, and R. Johnson, Freeze Drying of Biologics. In Encyclopedia of Pharmaceutical Science and Technology, 4th Ed. Taylor and Francis: New York, 1673-1722, 2013). It may be noted that a rigid glass, characterized by a high viscosity and low mobility, forms below the Tg′. When the Tg′ temperature is exceeded during the drying step, a viscous flow of the freeze-concentrated solute results in cake collapse, leading to a loss of the microstructure created by freezing.
the term “pellets” refers to particles with a tendency to be round.

The present invention relates to spray freeze-drying (SFD) processes. Such SFD processes may be used for the lyophilization of liquid pharmaceutical formulations including LNPs encapsulating mRNA, in particular mRNA vaccine formulations.

Spray freeze-drying (SFD) is a cutting-edge technology that can convert solutions (active pharmaceutical ingredients, excipients, and their combinations as pharmaceutical formulations) to bulk free-flowing dried pellets with cycle durations that, in general, are shorter than conventional vial freeze-drying (VFD). The present SFD process encompasses two main steps: (i) downward spraying of the solution into a cold gaseous environment to form sub-millimeter frozen droplets or pellets and (ii) stationary or dynamic vacuum drying in a rotary drum or other vacuum drying systems based on temperature-controlled surfaces in contact with the frozen droplets. Herein, the terms droplets and pellets may be used interchangeably.

Preferably, according to the invention, the SFD process comprises the sequential steps of:
spray freezing the liquid pharmaceutical formulation, in a column with temperature-controlled walls, the temperature of which is controlled by means of a cooling agent maintained at temperatures between −100 and −190° C., so as to obtain frozen pellets;
transferring the frozen pellets to a vacuum drying chamber; and
drying the frozen pellets within the vacuum drying chamber at a pressure no higher than 1000 microbar, the pellets being heated at a controlled temperature within the vacuum drying chamber.

The frozen pellets are heated within the vacuum drying chamber by direct contact with a temperature-controlled surface provided within the vacuum drying chamber.

Preferably, the temperature of the temperature-controlled surface within the vacuum drying chamber is varied within a range of 70° C. to +60° C., preferably of −45° C. to +50° C.

Optionally, the SFD process comprises, before the drying step, a pre-drying step that includes heating the frozen pellets to an annealing temperature greater than the glass transition temperature of the freeze concentrate (Tg′), but below the ice melting temperature.

The inventors have found that the inclusion of an annealing step in the methods of the invention can have the advantageous effects of
decreasing the duration of drying, by increasing the size of the ice crystals and consequently affecting the specific surface area and porosity of the dried pellets (the sugar and buffer matrix) containing the LNPs; and/or
maintaining the Z-average and polydispersity index (PDI) of the LNPs after spray freeze-drying and reconstitution, which are observed to remain similar to the pre-lyophilization LNP Z-average and PDI values.

Inclusion of an annealing step may be particularly advantageous if a shorter drying time is required and colloidal properties of LNPs need to be controlled.

Preferably, the annealing temperature according to the methods of the invention is 2° C. greater than the glass transition temperature of the freeze concentrate (Tg′) and 1° C. below the ice melting temperature.

Preferably, the annealing temperature according to the methods of the invention is 5° C. greater than the glass transition temperature of the freeze concentrate (Tg′) and 2° C. below the ice melting temperature.

Preferably, the annealing temperature according to the methods of the invention is 10° C. greater than the glass transition temperature of the freeze concentrate (Tg′) and 3° C. below the ice melting temperature.

Preferably, the annealing temperature according to the methods of the invention is 20° C. greater than the glass transition temperature of the freeze concentrate (Tg′) and 3° C. below the ice melting temperature.

Preferably, the annealing temperature according to the methods of the invention is 30° C. greater than the glass transition temperature of the freeze concentrate (Tg′) and 3° C. below the ice melting temperature.

In particular, the glass transition temperature of the freeze concentrate (Tg′) is measured by Differential Scanning Calorimetry.

In particular, the ice melting temperature of the freeze concentrate is measured by Differential Scanning Calorimetry.

Preferably, the drying step may be performed in a rotary drum, wherein the temperature-controlled surface is formed by the internal surface of the rotary drum within the vacuum drying chamber. Using such a rotary drum was found to provide a higher throughput as compared to other methods of performing the drying step.

Alternatively, the drying step may be performed on a static shelf or a set of static shelves, wherein the temperature-controlled surface is formed by the surface of said static shelve(s) within the vacuum drying chamber.

As an alternative to heating the frozen pellets by direct contact with a temperature-controlled surface, the pellets may be heated within the vacuum drying chamber by contactless heating, preferably by means of electromagnetic radiation, in particular infra-red or radio-frequency (e.g. microwave) radiation. Any other means of delivering energy for non-destructive product dehydration may be used.

Suitable lipids for use in a formulation that can be lyophilized by the method of the invention are described below.

The lipid component of a LNP may include, for example, a cationic lipid, a phospholipid (such as an unsaturated lipid, e.g., DOPE or DSPC), a structural lipid (e.g. polyethylene glycol (PEG) lipid or cholesterol), or any combination thereof. The elements of the lipid component may be provided in specific fractions.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic and other elements (e.g., lipids) in a LNP may also vary. In some embodiments, the wt/wt ratio of the lipid component to a therapeutic and/or prophylactic in a LNP may be from about 5:1 toabout 60:1, such as 5:1, 6:1, 7:1,8:1,9:1, 10:1, 11:1, 12:1, 13:1, 14:1, 15:1, 16:1, 17:1, 18:1, 19:1, 20:1, 25:1,30:1,35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

In some embodiments, the ionizable lipid is a compound of Formula (IL-I):

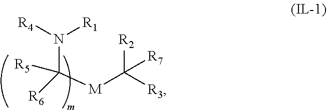

or their N-oxides, or salts or isomers thereof, wherein: Ri is selected from the group consisting of C5-30 alkyl, C5-20 alkenyl, −R*YR″, —YR″, and —R″M′R′; R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle; R4 is selected from the group consisting of hydrogen, a C3-6 carbocycle, —(CH2)nQ, —(CH2)nCHQR, —CHQR, —CQ(R)2, and unsubstituted C1-6 alkyl, where Q is selected from a carbocycle, heterocycle, —OR, —0(CH₂)nN(R)2, —C(O)OR, —OC(O)R, —CX3, —CX2H, —CXH2, —CN, —N(R)2, —C(O)N(R)2, —N(R)C(O)R, —N(R)S(0)₂R, —N(R)C(O)N(R)2, —N(R)C(S)N(R)2, —N(R)Re, N(R)S(0)₂R₈, —0(CH₂)nOR, —N(R)C(═NR9)N(R)₂, —N(R)C(═CHR9)N(R)₂, —OC(O)N(R)_{2J}—N(R)C(O)OR, —N(OR)C(O)R, —N(OR)S(0)₂R, —N(OR)C(O)OR, —N(OR)C(O)N(R)₂, —N(OR)C(S)N(R)2, —N(OR)C(═NR9)N(R)₂, —N(OR)C(═CHR9)N(R)₂, —C(═NR9)N(R)₂, —C(═NR9)R, —C(O)N(R)OR, and —C(R)N(R)2C(O)OR, and each n is independently selected from 1, 2, 3, 4, and 5; each R5 is independently selected from the group consisting of C1-3 alkyl, C2-3 alkenyl, and H; each Re is independently selected from the group consisting of C1-3 alkyl, C2-3 alkenyl, and H; M and M′ are independently selected from —C(O)O—, —OC(O)—, —OC(O)-M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C1-13 alkyl or C₂₋₁₃ alkenyl; R7 is selected from the group consisting of C1-3 alkyl, C2-3 alkenyl, and H; Re is selected from the group consisting of C3-6 carbocycle and heterocycle; R9 is selected from the group consisting of H, CN, NO2, Ci-6 alkyl, —OR, —S(0)2R, —S(O)2N(R)2, C2-6 alkenyl, C3-6 carbocycle and heterocycle; each R is independently selected from the group consisting of C1-3 alkyl, C₂-3 alkenyl, and H; each R′ is independently selected from the group consisting of Ci-is alkyl, C₂-is alkenyl, —R*YR″, —YR″, and H; each R″ is independently selected from the group consisting of C3-15 alkyl and C3-15 alkenyl; each R* is independently selected from the group consisting of Ci-i₂ alkyl and C₂₋ᵢ₂ alkenyl; each Y is independently a C3-6 carbocycle; each X is independently selected from the group consisting of F, C, Br, and I; and m is selected from 5, 6, 7, 8, 9, 10, 11, 12, and 13; and wherein when R₄ is —(CH₂)ₙQ, —(CH₂)nCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

The lipid component of a lipid nanoparticle composition may include one or more molecules comprising polymers, such as polyethylene glycol PEG)-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerCl4 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid. In some embodiments, the PEG-modified lipids are a modified form of PEG DMG. In some embodiments, the PEG-modified lipid is PEG lipid with the formula (IV):

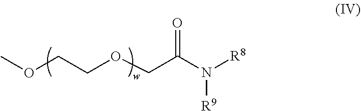

wherein R⁸ and R⁹ are each independently a straight or branched, saturated or unsaturated alkyl chain containing from 10 to 30 carbon atoms, wherein the alkyl chain is optionally interrupted by one or more ester bonds, and w has a mean value ranging from 30 to 60.

### BRIEF DESCRIPTION OF THE FIGURES

The invention will now be further illustrated by the following Example, with reference to the Figures listed below, wherein:

FIG. 1 is a schematic view of a spraying column used in a process of the invention:

FIG. 2 represents the evolution of the cooling gas inside the spraying column;

FIG. 3 illustrates data generated in relation to an Example of drying with rotary drum;

FIG. 4 illustrates data generated in relation to an Example of shelf-based drying of spray frozen pellets with annealing;

FIG. 5 illustrates data generated in relation to an Example of shelf-based drying of frozen pellets without annealing;

FIG. 6 illustrates data generated in relation to an Example of shelf-based drying of a single layer of frozen pellets without annealing;

FIG. 7 shows an image of a single layer of frozen pellets during drying in the Example referred to in FIG. 6;

FIG. 8 shows images of vial freeze-dried cakes and spray freeze-dried pellets;

FIG. 9 represents the evolution of moisture content in lyophilized formulations 1 and 2 (300 mM and 600 mM sucrose), samples stored at 5° C. (with VFD data generated only at initial timepoint t0);

FIG. 10 represents the evolution of colloidal stability of LNPs post storage of lyophilized formulation 1 (300 mM sucrose) at 5° C. (left) and 25° C. (right);

FIG. 11 represents the evolution of colloidal stability of LNPs post storage of lyophilized formulation 2 (600 mM sucrose) at 5° C. (left) and 25° C. (right);

FIG. 12 represents the evolution of % encapsulation and concentration of mRNA post storage of lyophilized formulation 1 (300 mM sucrose) at 5° C. (left) and 25° C. (right);

FIG. 13 represents the evolution of % encapsulation and concentration of mRNA post storage of lyophilized formulation 2 (600 mM sucrose) at 5° C. (left) and 25° C. (right);

FIG. 14 represents the evolution of mRNA integrity post storage of lyophilized formulation 1 (300 mM sucrose) at 5° C. (left) and 25° C. (right);

FIG. 15 represents the evolution of mRNA integrity post storage of lyophilized formulation 2 (600 mM sucrose) at 5° C. (left) and 25° C. (right); and

FIG. 16 illustrates In-vitro expression (IVE) post storage of lyophilized formulations 1 and 2 (300 mM and 600 mM sucrose) at 5° C.

### EXAMPLE

The following Example illustrating the invention corresponds to the lyophilization of a specific mRNA vaccine formulation, in this particular case a Flu modRNA (Washington strain) drug product (DP). The influenza modRNA immunogenic composition is comprised of one or more nucleoside-modified mRNAs that encode the full-length HA glycoprotein derived from seasonal human influenza strains. The specific construct (HA modRNA) is the only active ingredient in the immunogenic composition. In addition to the codon-optimized sequence encoding the antigen, the RNA contains common structural elements optimized for mediating high RNA stability and translational efficiency (5′-cap, 5′UTR, 3′-UTR, poly(A)-tail). The RNA does not contain any uridines; instead of uridine, the modified N1-methylpseudouridine is used in RNA synthesis. The 5′-cap analog (m₂⁷·3′^{—OMe}Gppp(m₁2′^{—O})ApG) for production of the RNA containing a cap1 structure is shown below.

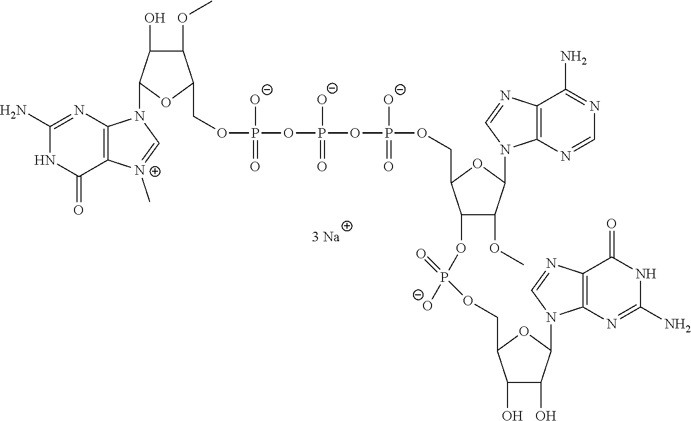 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O ; "3 Na"]

The above structure corresponds to Trilink's CleanCap AG (3′OMe)-m₂⁷,3′-OGppp (m₁2′-O)ApG.

Lipid nanoparticles were prepared and tested according to the general procedures described in U.S. Pat. No. 9,737,619 (PCT Pub. No. WO2015/199952) and U.S. patent Ser. No. 10/166,298 (PCT Pub. No. WO 2017/075531) and PCT Pub. No. WO2020/146805.

In the Example, the mRNA vaccine formulation includes LNPs encapsulating mRNA, that are dispersed in a matrix suitable for freeze-drying.

The Example and the Figures are provided for illustrative purpose only and should not be construed as limiting the scope of the invention.

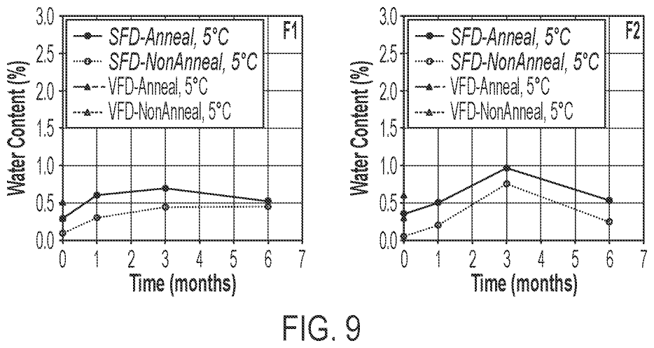

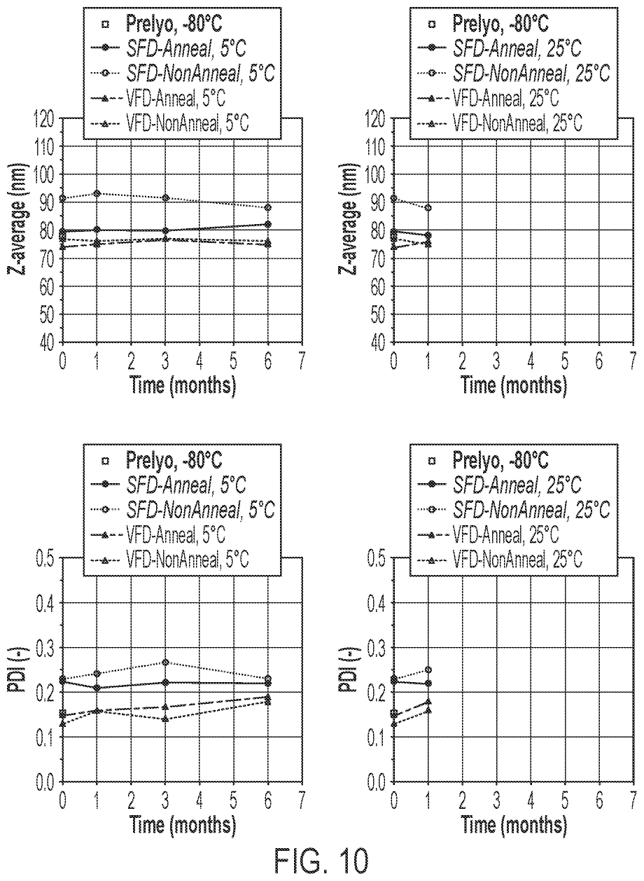

A short-term (6-month) study was designed to assess the stability of the lyophilized formulation using SFD and conventional VFD: FIG. 9-16. The effects of sucrose concentration as a cryoprotectant and lyoprotectant (300 mM vs. 600 mM sucrose matrices) and annealing post-freezing on product attributes were also evaluated.

It was surprisingly found that, in particular and beyond the advantage of a shorter drying time, the product attributes of SFD pellets from the mRNA formulation demonstrated the following advantageous properties:
excellent flowability of the dry pellets allowing to fill any types of containers (vials, blisters, dual chamber cartridges/syringes, large bottles, etc.,);
dosing and filling flexibilities, which can be defined and conducted post lyophilization.

### A. Spray-Freezing

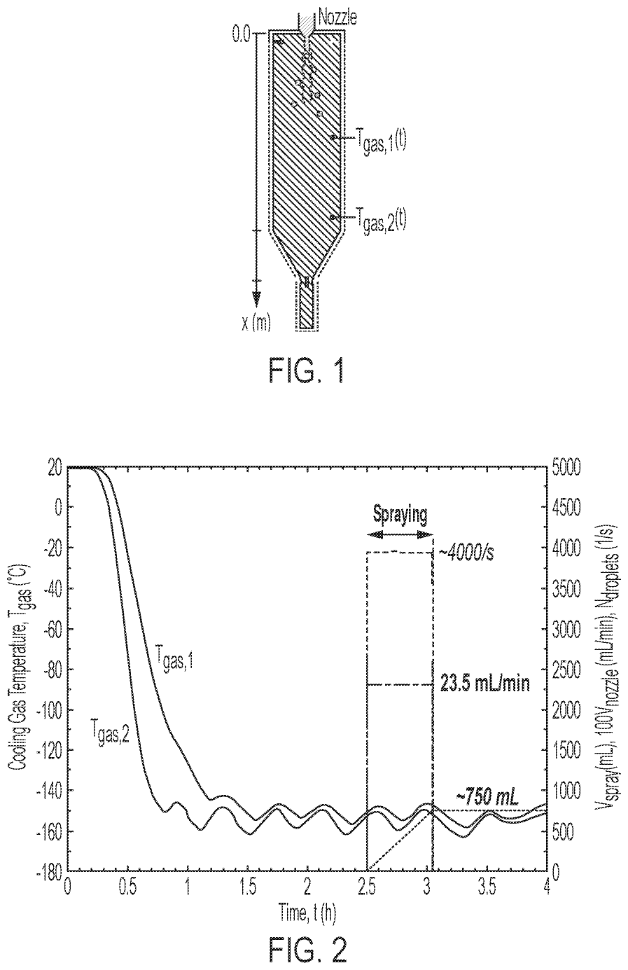

At the top of a spraying column, such as shown on FIG. 1, with temperature-controlled walls, the liquid formulation at a temperature equal to or greater than refrigerated temperature (2° to 8° C. range) is pumped through a prilling nozzle that generates monodispersed droplets via laminar jet break-up. The cooling gas inside the column is maintained at a target temperature (within −100° to −190° C. range) by means of an annular cooling jacket (LN2 or a mixture of LN2 and gaseous N2). Nozzle orifices in the range of 0.2 to 0.5 mm are utilized to generate pellets or droplets with 0.4 mm to 1.0 mm diameter. The corresponding range of prilling frequencies is 500-6500 Hz with mass flow rates between 5 and 50 g/min. Deflection jet gas flow rates of ≤0.3 m³/h are utilized to disperse droplets and improve the spray-freezing efficiency. The spray-frozen pellets or droplets are collected at the bottom of the freezing column for subsequent drying or storage. FIG. 2 provides an example of the current lab-scale spray-freezing process (Sebastião IB, Bhatnagar B, Tchessalov S. A Kinetic Model for Spray-Freezing of Pharmaceuticals, Journal of Pharmaceutical Sciences 110 (2021) 2047-2062).

In FIG. 2, T_{gas,1} and T_{gas,2} represent the evolution of the cooling gas inside the freezing column. In this example, 750 mL of a liquid formulation was sprayed at a flow rate of 23.5 mL/min and a nominal prilling frequency of 4000 droplets per second. Highly-viscous solutions (e.g., >5 cP) can be sprayed at higher temperatures—leading to improved pumping and spraying performances—without significant increase in the freezing column size (Sebastiso I B, Bhatnagar B, Tchessalov S, Ohtake S, Plitzko M, Luy B, Alexeenko A, Bulk Dynamic Spray Freeze-Drying Part 2: Model-Based Parametric Study for Spray-Freezing Process Characterization, Journal of Pharmaceutical Sciences 108 (2019), 2075-2085).

The spray freeze-dried pellets are sub-millimeter sized (e.g., 200-1000 micrometer) and, in the context of present invention, they contain the lipid nanoparticles (preferably with a Z-average in the range of 20 to 150 nm).

### B.1 Rotary Vacuum Drying

In the case of the rotary vacuum dryer, the drying process is initiated by pre-cooling the drum walls to a temperature below −45° C. or respective glass transition temperature of the freeze concentrate of formulation. Once this target condition is achieved, the spray-frozen pellets are transferred to the drum which then starts to rotate at speeds between 0 and 5 RPM. At this point, annealing of the frozen pellets at or above the glass transition temperature of the matrix can be performed as needed.

Omission of the annealing step in the context of the present invention may be contemplated if no impact of spray freeze-drying on the LNP Z-average and PDI is expected and/or if pellets become more friable, leading to generation of dust during processing and reduction of yield.

During annealing, a frozen solution is heated to a temperature greater than the glass transition temperature of the freeze concentrate (Tg′), but below the eutectic melting, secondary melting, or ice melting temperature. Annealing has been shown to increase the ice crystal size and sublimation rate and decreases the inter-vial heterogeneity in drying rates during lyophilization (Bhatnagar B, Tchessalov S., Lewis L, and Johnson R, Freeze-drying of Biologics. In: Encyclopedia of Pharmaceutical Science and Technology, 4th edition, Publisher Taylor & Francis, 2013:1673-1722; Searles J A, Carpenter J F, Randolph T W. Annealing to optimize the Primary Drying Rate, reduce Freezing-induced Drying Rate Heterogeneity, and determine Tg′ in Pharmaceutical Lyophilization. Journal of Pharmaceutical Sciences 90 (2001) 872-887; Tang X, Pikal M J 2004. Design of Freeze-Drying Processes for Pharmaceuticals: Practical Advice. Pharm Res 21(2):191-200).

The next step is to reduce the pressure in the drying chamber that encompasses the drum to no higher than 1000 microbar (750 mTorr), preferably at pressures no higher than 500 microbar (375 mTorr). Primary drying is then performed by a sequence of stepwise or continuous changes in various drying parameters, including chamber pressure, drum wall temperature, drum speed, and power of infrared (IR) heater. The target final set of primary drying parameters is held until end of sublimation is indicated by convergence of Pirani gauge and capacitance manometer (CM) pressure readings. During secondary drying, the residual water in the partially dried droplets or pellets is removed via desorption by increasing the drum temperature and (or) IR power.

The primary and secondary drying parameters can vary between 0 to 1000 microbar (0 to 750 mTorr), −70° C. to +60° C., 0 to 10 RPM, and 0 to 25000 W.

Preferably, these ranges are respectively 0 to 500 microbar (0 to 375 mTorr), −45° C. to +50° C., 0 to 5 RPM, and 0 to 15000 W.

After completion of secondary drying, which is also indicated by Pirani gauge and CM convergence, the bulk pellets (specific surface area ≥2 m²/g) are unloaded to a collection vessel under a dry atmosphere for either storage or subsequent filling into target containers.

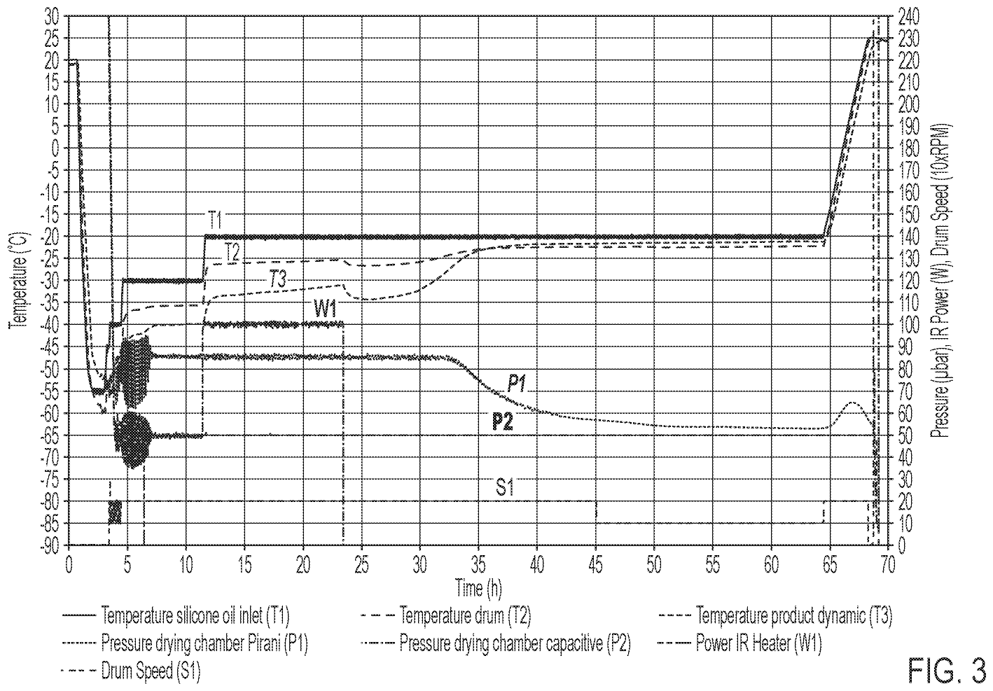

FIG. 3 provides an example of the current lab-scale drying process in rotary drum.

The frozen pellets are herein loaded into the pre-cooled drum with surface temperature (T2) below −55° C. The drum temperature is controlled by means of a built-in annular cooling jacket and respective inlet temperature of the coolant fluid (T1), which in this case is silicone oil. A temperature sensor attached to drum wall indicates the bulk product, i.e., pellets, temperature (T3). Post loading and conditioning of the frozen pellets, the chamber is sealed, and vacuum is initiated. In the present example, a pressure of 50 microbar (37.5 mTorr) was held during the entire drying process as indicated by the CM reading (P2). Completion of primary and secondary drying are indicated by convergence of Pirani gauge (P1) and CM values. Different setpoints for the infrared radiator power (W1) and drum speed (S1) were utilized throughout the process to meet the target product temperature history and total drying time.

### B.2 Alternative Drying of Frozen Pellets

As an alternative drying process, the spray-frozen pellets can be transferred to any vacuum drying system that utilizes temperature-controlled surfaces, e.g., a conventional shelf-based lyophilizer or set of cascading shelves (available from IMA-Group. LYNFINITY: Continuous aseptic spray-freeze-drying. 2020). The range of respective primary and secondary drying parameters can vary between 0 to 1000 microbar (0 to 750 mTorr) for the chamber pressure, −70° C. and +60° C. for the temperature-controlled surfaces. Preferably, these ranges are respectively 0 to 500 microbar (0 to 375 mTorr) and −45° C. to +50° C. The end point of primary and secondary drying can be identified by any known means including, but not limited to, Pirani-CM convergence, pressure rise methods, tunable diode laser absorption spectroscopy (TDLAS), mass spectrometers, IR, and near-IR detectors.

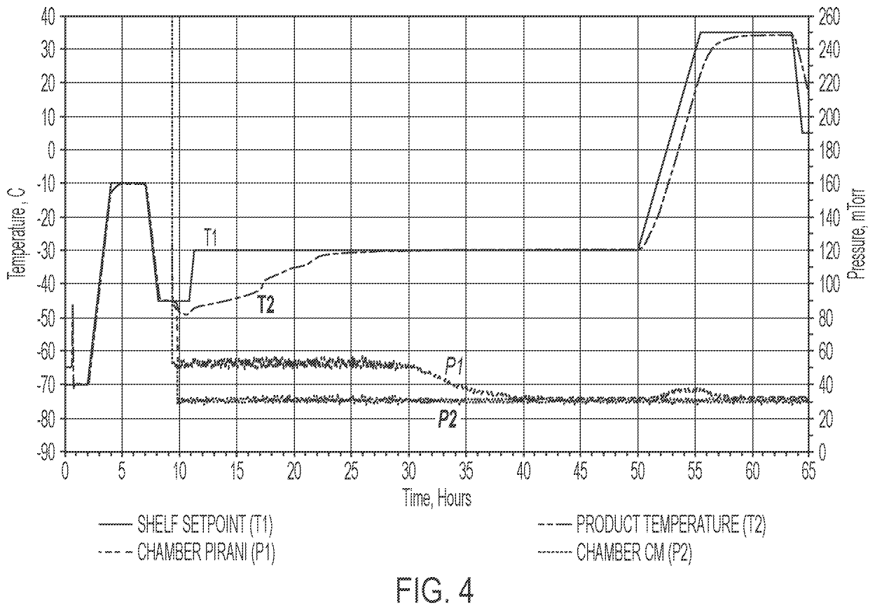

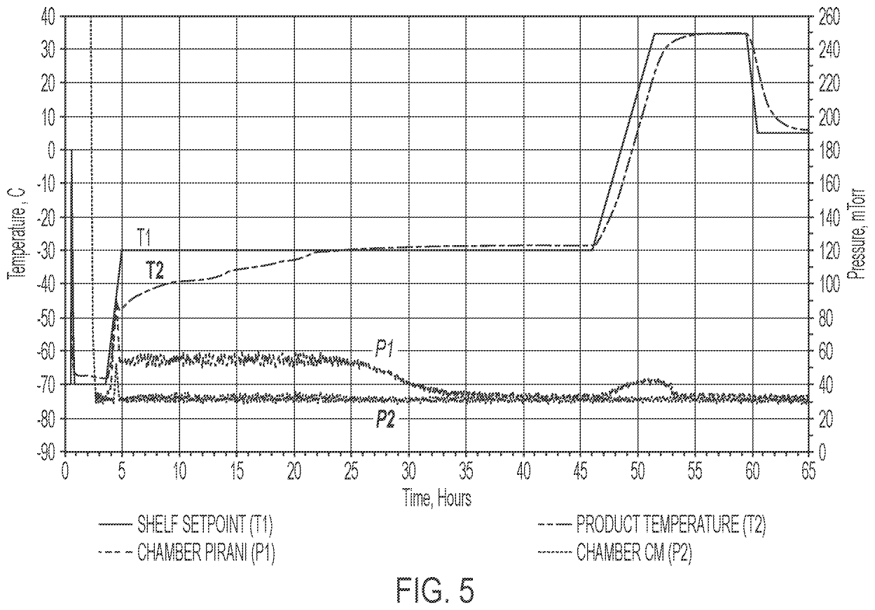

FIGS. 4 and 5 provide examples of the shelf-based drying process in conventional freeze-dryers with and without annealing, respectively.

In these figures, T1 represents the temperature setpoint of the temperature-controlled surfaces—the shelves of a lyophilizer in this case—utilized to dry the spray-frozen pellets. The respective product temperature is indicated by T2. Post loading and conditioning of the frozen pellets, the chamber is sealed, and vacuum is initiated. In this example, a pressure of 40 microbar (30 mTorr) was held during the entire drying process as indicated by the CM reading (P2). Completion of primary and secondary drying are indicated by convergence of Pirani gauge (P1) and CM values.

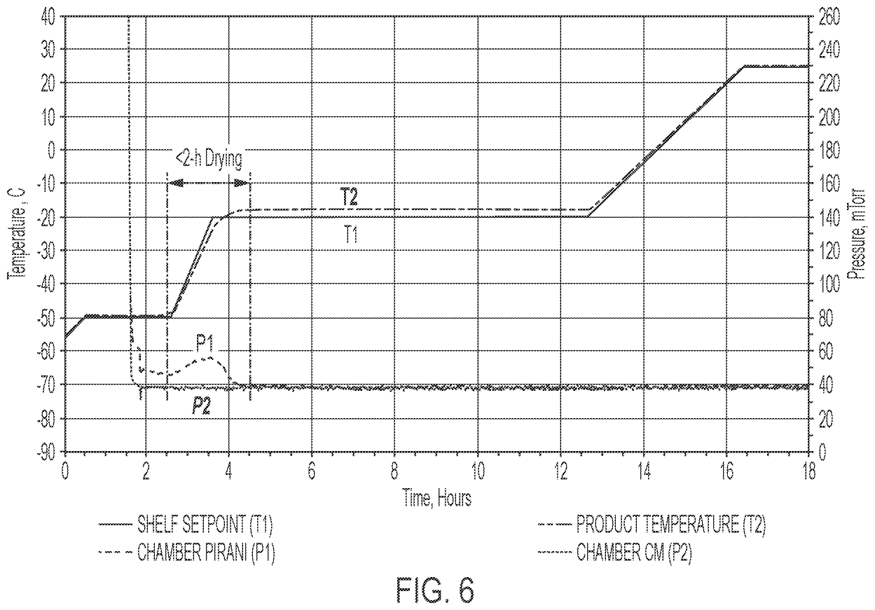

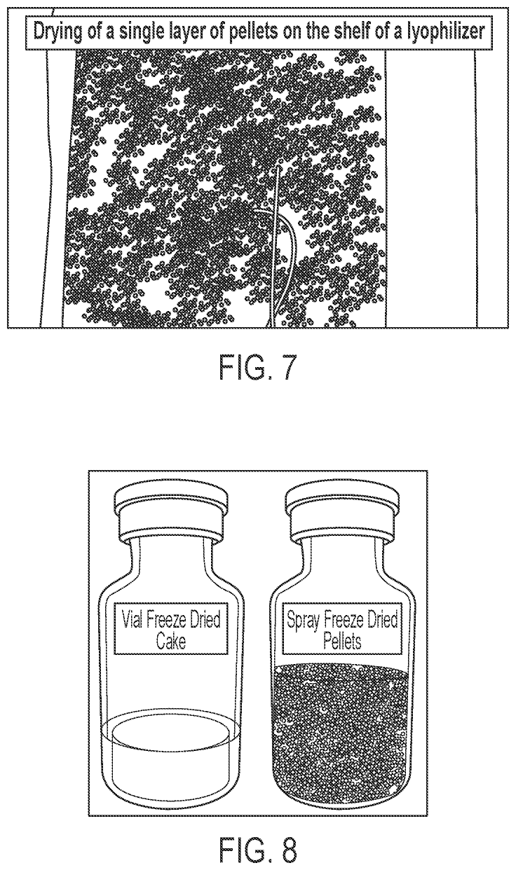

The drying time of spray-frozen pellets can be reduced by decreasing the bed thickness of the bulk product. As illustrated on FIG. 6, drying of one layer of frozen pellets can be completed in less than 2 hours.

### C. Analytical Assessment of Product Quality

Lipid nanoparticles (LNPs) are characterized by a variety of methods. For example, microscopy (e.g., transmission electron microscopy or scanning electron microscopy) is utilized to examine the size and morphology of LNP. Table 1 summarizes analytical techniques that are utilized for characterization of Iyophilized LNPs.

TABLE 1
Examples of analytical methods for characterization
of lyophilized LNPs.

| Product Attributes | Analytical Procedure |
|---|---|
| Residual moisture | Karl Fischer coulometric |
| | titration and NIR |
| Thermal behavior (melting | Differential scanning |
| temperature, glass | calorimetry |
| transition temperature) | |
| Phase behavior (crystalline/ | X-ray diffraction |
| amorphous phases) | |
| LNP size and polydispersity | Dynamic light scattering |
| RNA Encapsulation and content | Fluorescence assay |
| RNA integrity | Capillary gel electrophoresis |
| LNP morphology | Electron microscopy |
| In vitro expression | Cell-based flow cytometry |
| Lipids content | HPLC-CAD |
| Specific surface area | BET gas adsorption-desorption |

Dynamic light scattering or potentiometry (e.g., potentiometric titrations) may be used to measure zeta potentials. Dynamic light scattering (DLS) is conventionally utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index (PDI), and zeta potential.

Preferably the Z-average of the LNPs post drying measured using DLS according to the invention is in the range of 20 to 180 nm, more preferably in the range of 30 to 150 nm, and most preferably in the range of 40 to 120 nm.

Preferably the PDI of the LNPs post drying measured using DLS according to the invention is in the range of 0.01 to 0.5, more preferably in the range of 0.05 to 0.4 and most preferably in the range of 0.1 to 0.3.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%).

Preferably, the efficiency of encapsulation post drying according to the present invention is greater than 70%, more preferably greater than 80%, and most preferably greater than 90%.

The encapsulation efficiency may be measured, for example, by comparing the amount of therapeutic and/or prophylactic in a medium containing the lipid nanoparticle before and after breaking up the lipid nanoparticle with one or more organic solvents or detergents. Fluorescence measured using a Tecan Fluorescent Plate Reader (Tecan Group Ltd, Mannedorf, Switzerland) is used to determine the amount of free therapeutic and/or prophylactic (e.g., RNA) in a solution.

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity. Fragment Analyzer (FA) systems are multiplexed capillary gel electrophoresis (CGE) instruments that can perform high-throughput separation and quantification of mRNA (and other RNAs). The percent integrity of mRNA is determined using an FA Automated CGE System (AgilentTechnologies Inc, Agilent, California, USA).

Preferably the RNA has an integrity post drying of at least 50%, preferably of at least 60%, more preferably of at least 70%, more preferably of at least 80%, most preferably of at least 90%.

The present study was designed to assess the stability of Flu mRNA (Washington strain) DP lyophilized with SFD and VFD technologies. The effects of sucrose concentration as cryoprotectant and lyoprotectant (300 mM vs. 600 mM sucrose matrices) and annealing on product attributes were also evaluated. Two LNP-based formulations (F1 and F2, Table 2) suitable for use in the context of the present invention were assessed and are described in Table 2.

TABLE 2
Formulation composition prior to VFD and SFD and dry samples
weights per vial (cake from VFD and pellets from SFD).

| | | | Cake | Pellets |
|---|---|---|---|---|
| | Flu mRNA, | | weight | weight |
| Formulation | mg/mL | Matrix composition | (VFD) | (SFD) |
| F1 | 0.1 | 300 mM (10.3% w/v) | 32 mg | 32 mg |
| | | Sucrose, 10 mM Tris, | | |
| | | pH 7.4 | | |
| F2 | 0.1 | 600 mM (20.5% w/v) | 63 mg | 63 mg |
| | | Sucrose, 10 mM Tris, | | |
| | | pH 7.4 | | |

The dried samples (FIG. 8, representative images shown) were enrolled in a stability study at 2-8° C. (herein, indicated as 5° C. for brevity) and 25° C. to assess the impact of storage conditions on residual moisture content, average nanoparticle size, PDI, mRNA encapsulation, concentration, integrity, and in-vitro expression (IVE). Since a limited number of vials were available for storage at 25° C., unless stated otherwise, only 1 month data were generated for this arm of the study. At the respective timepoints, samples were reconstituted to the pre-lyophilization (prelyo) concentration of 0.1 mg/mL mRNA using USP 0.9% w/v saline or sterile water for injection (sWFI).

In the plots referred to below and presented on the Figures, the mRNA-containing formulations 1 and 2 are simply referred to as 300- and 600-mM sucrose formulations, respectively. Likewise, the lyophilized samples with and without annealing are listed as “Anneal” and “NonAnneal”.

### C.1 Residual Moisture Content

The residual moisture content in VFD and SFD samples was determined with a coulometric Karl-Fischer titrator (Photovolt Aquatest™ 2010 Karl Fischer Coulometric Moisture Titrator, Photovolt Instruments, St. Louis Park, MN). FIG. 9 shows the evolution of the moisture content for SFD samples stored for 6 months (6M) at 5° C. The VFD data was generated only at the initial timepoint (t0). These data suggest that the present SFD samples with and without annealing have similar rate of moisture uptake.

Preferably the dried composition has a residual water content achieved by the methods of the invention below 4%, more preferably below 2%, and most preferably below 1% about 2 weeks to about 1 month, 2 months, 3 months, 4 months, 5 months, 6 months, 9 months, 1 year, or 2 years after storage as a dried composition at temperatures less than or equal to refrigerated storage (“refrigerated storage” is defined as the 2° to 8° C. temperature range).

### C.2 Colloidal Stability of LNPs

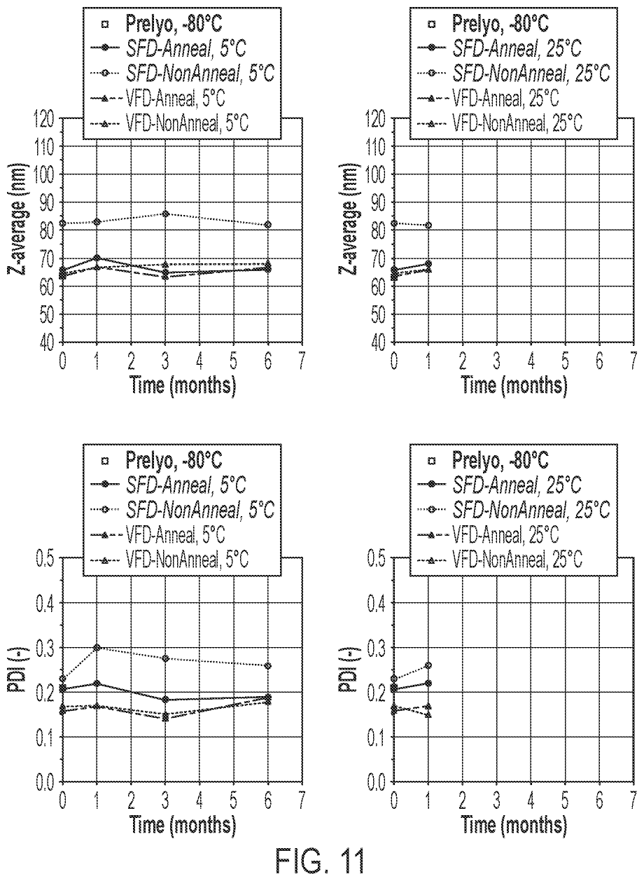

The colloidal stability of the LNPs in lyophilized VFD and SFD samples was determined via DLS assay. FIGS. 10 and 11 show the evolutions of the average nanoparticle sizes (Z-average) and PDIs for formulations 1 and 2, respectively.

The colloidal stability of the unannealed and annealed VFD formulations is comparable and also similar to the SFD formulations that included annealing. In the absence of annealing, the SFD formulations exhibited larger LNP size and PDI (FIGS. 10 and 11).

Preferably the Z-average of the LNPs post drying achieved by the methods of the invention is in the range of 20 to 180 nm, more preferably in the range of 30 to 150 nm, and most preferably in the range of 40 to 120 nm about 2 weeks to about 1 month, 2 months, 3 months, 4 months, 5 months, 6 months, 9 months, 1 year, or 2 years after storage as a dried composition at temperatures less than or equal to refrigerated storage (2° to 8° C. range).

Preferably, these LNP sizes are obtained when including an annealing step in the methods according to the invention.

Preferably the PDI of the LNPs post drying achieved by the methods of the invention is in the range of 0.01 to 0.5, more preferably in the range of 0.05 to 0.4 and most preferably in the range of 0.1 to 0.3 about 2 weeks to about 1 month, 2 months, 3 months, 4 months, 5 months, 6 months, 9 months, 1 year, or 2 years after storage as a dried composition at temperatures less than or equal to refrigerated storage (2⁰ to 8° C. range). Preferably, these PDI values are obtained when including an annealing step in the methods according to the invention.

C.3 Encapsulation and Concentration of mRNA

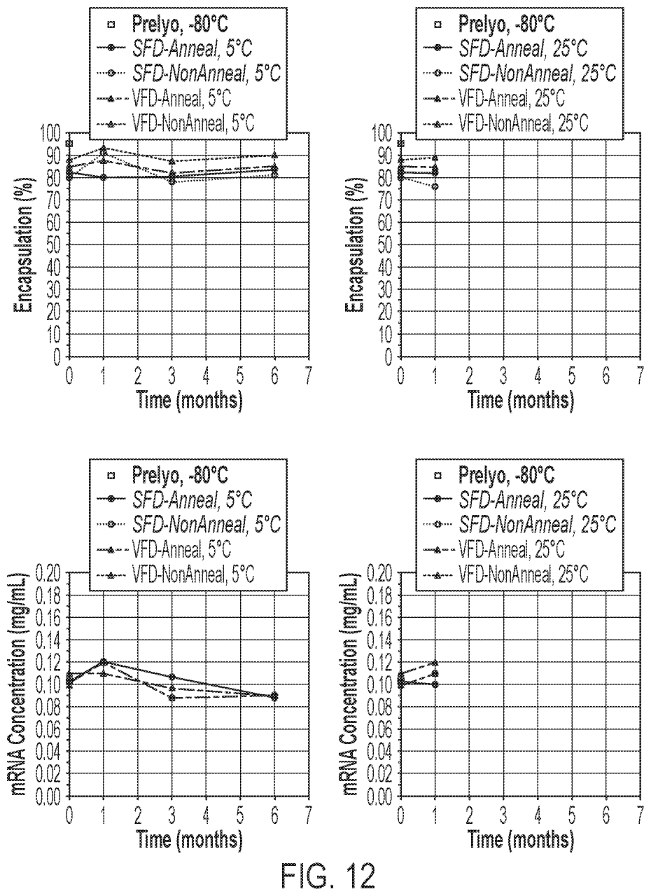

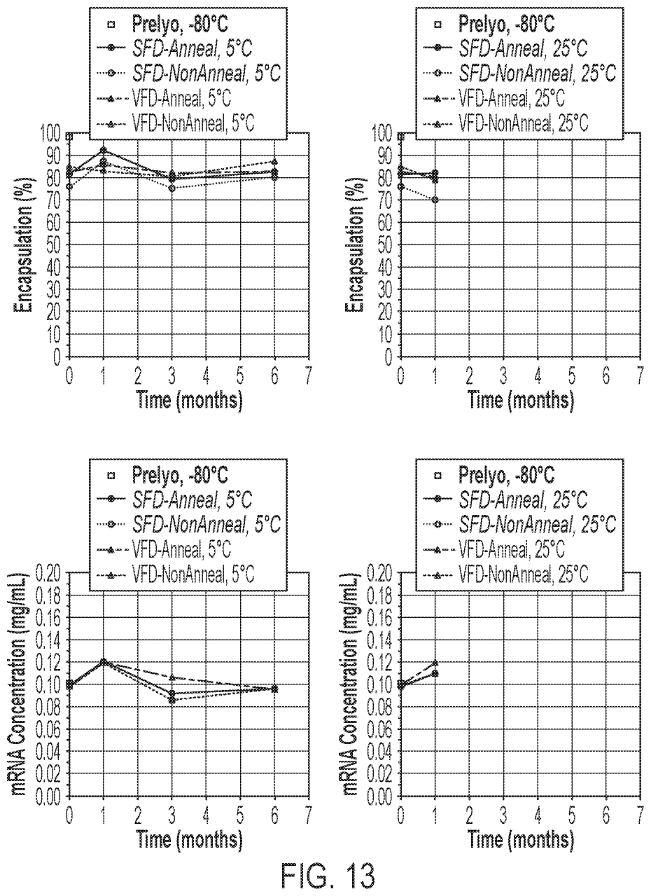

The % encapsulation and concentration of mRNA in lyophilized VFD and SFD samples were determined via a RiboGreen® assay. FIGS. 12 and 13 provide the evolutions of these two attributes for formulations 1 and 2, respectively. There was no change in % encapsulation and mRNA concentration of VFD and SFD formulations during storage at both temperatures (i.e. at 5° C. and 25° C., respectively).

Preferably the efficiency of encapsulation post drying achieved by the methods of the invention is greater than 70%, more preferably greater than 80%, and most preferably greater than 90% about 2 weeks to about 1 month, 2 months, 3 months, 4 months, 5 months, 6 months, 9 months, 1 year, or 2 years after storage as a dried composition at temperatures less than or equal to refrigerated storage (2° to 8° C. range).

Preferably the mRNA concentration post drying achieved by the methods of the invention of about 2 mg/mL, preferably of at least about 0.5 mg/mL, more preferably of at least about 0.1 mg/mL, most preferably of at least about 0.001 mg/mL about 2 weeks to about 1 month, 2 months, 3 months, 4 months, 5 months, 6 months, 9 months, 1 year, or 2 years after storage as a dried composition at temperatures less than or equal to refrigerated storage (2° to 8° C. range).

C.4 Integrity of mRNA

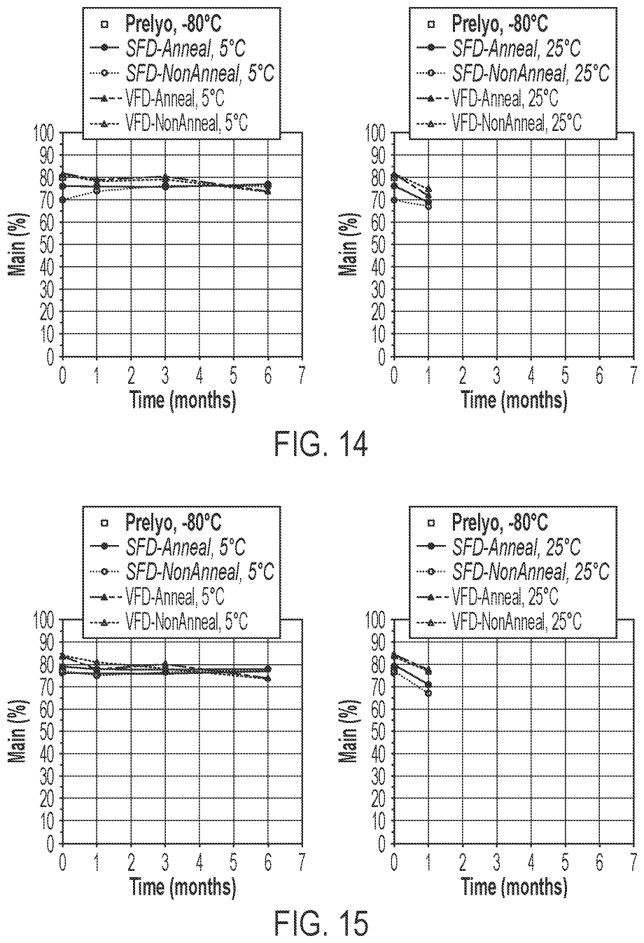

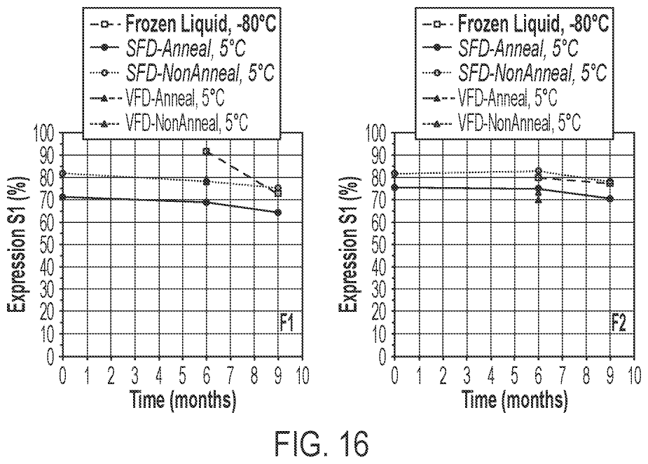

The mRNA integrity in lyophilized VFD and SFD samples was determined via a fragment analysis (FA) assay. FIGS. 14 and 15 present the evolutions of the percentage of mRNA integrity for formulations 1 and 2, respectively. There was no change in % RNA integrity of VFD and SFD formulations during storage at 5° C. During storage at 25° C., a decrease in % RNA integrity was observed in VFD and SFD formulations.

Preferably the RNA has an integrity post drying achieved by the methods of the invention of about 50%, preferably of at least about 60%, more preferably of at least about 70%, more preferably of at least about 80%, most preferably of at least about 90% about 2 weeks to about 1 month, 2 months, 3 months, 4 months, 5 months, 6 months, 9 months, 1 year, or 2 years after storage as a dried composition at temperatures less than or equal to refrigerated storage (2° to 8° C. range).

### C.5 In-Vitro Expression

The in-vitro expression (IVE) of mRNA in lyophilized VFD and SFD samples was determined via a cell-based assay with fluorescence-activated cell sorter (FACS). FIG. 16 shows the % IVE expression profiles overtime for formulations 1 and 2, respectively. The % expression did not decrease during storage of VFD and SFD formulations at 5° C.

Preferably the % IVE achieved by the methods of the invention is at least 30%, preferably at least about 40%, more preferably at least about 50%, more preferably at least about 60%, more preferably at least about 70%, and most preferably at least about 80% about 2 weeks to about 1 month, 2 months, 3 months, 4 months, 5 months, 6 months, 9 months, 1 year, or 2 years after storage as a dried composition at temperatures less than or equal to refrigerated storage (2° to 8° C. range).

### C.6 Effects of Sucrose Concentration as a Cryoprotectant and Lyoprotectant

Preferred sucrose concentrations used in the formulations and processes of the present invention are in the range of 5 to 60%, more preferably in the range of 5 to 25%, and most preferably in the range of 10 to 20%.

In conclusion of the above-described experiments, it has been shown that an SFD process is suitable for lyophilizing a liquid pharmaceutical formulation including lipid nanoparticles encapsulating mRNA. In particular, the data generated in those experiments suggests that product attributes of mRNA formulations subjected to SFD are comparable to those obtained with conventional VFD. Also, the data suggest that the inclusion of an annealing step, while providing certain advantageous effects (referred to in B.1), does not negatively affect the evaluated product attributes.

Therefore, the described SFD process can be advantageously utilized to achieve lyophilization for production of dried pharmaceutical products or product intermediates.

1. Use of a spray freeze-drying process for the lyophilization of a liquid pharmaceutical formulation including lipid nanoparticles encapsulating mRNA.

2. Use according to claim 1, wherein the spray freeze dying process comprises the sequential steps of:
spray freezing the liquid pharmaceutical formulation, in a column with temperature-controlled walls, the temperature of which is controlled by means of a cooling agent maintained at temperatures between −100 and −190° C., so as to obtain frozen pellets;
transferring the frozen pellets to a vacuum drying chamber; and
drying the frozen pellets within the vacuum drying chamber at a pressure no higher than 1000 microbar, the pellets being heated at a controlled temperature within the vacuum drying chamber.

3. Use according to claim 2, wherein the frozen pellets are heated within the vacuum drying chamber by direct contact with a temperature-controlled surface provided within the vacuum drying chamber.

4. Use according to claim 3, wherein the temperature-controlled surface is formed by the internal surface of a rotary drum within the vacuum drying chamber.

5. Use according to claim 4, wherein the rotation speed of the rotary drum is between 0 and 10 RPM, preferably between 0 and 5 RPM.

6. Use according to claim 3, wherein the temperature-controlled surface is formed by the surface of a static shelf or of a set of static shelves within the vacuum drying chamber.

7. Use according to claim 3, wherein the temperature of the temperature-controlled surface within the vacuum drying chamber is varied within a range of −70° C. to +60° C., preferably of −45° C. to +50° C.

8. Use according to claim 3, wherein the pressure within the vacuum drying chamber is varied between 0 to 1000 microbar (0 to 750 mTorr), preferably between 0 to 500 microbar (0 to 375 mTorr).

9. Use according to claim 2, wherein the frozen pellets are heated within the vacuum drying chamber by contactless heating.

10. (canceled)

11. Use according to claim 2, wherein the spray freeze dying process comprises, before drying the frozen pellets, a pre-drying step including heating the pellets to an annealing temperature greater than the glass transition temperature of the freeze concentrate (Tg′), but below the ice melting temperature.

12. Use according to claim 11, wherein the annealing temperature is 2° C. greater than the glass transition temperature of the freeze concentrate (Tg′) and 1° C. below the ice melting temperature, preferably 5° C. greater than the glass transition temperature of the freeze concentrate (Tg′) and 2° C. below the ice melting temperature, more preferably 10° C. greater than the glass transition temperature of the freeze concentrate (Tg′) and 3° C. below the ice melting temperature, more preferably 20° C. greater than the glass transition temperature of the freeze concentrate (Tg′) and 3° C. below the ice melting temperature, and still more preferably 30° C. greater than the glass transition temperature of the freeze concentrate (Tg′) and 3° C. below the ice melting temperature.

13. (canceled)

14. Process of lyophilization of a liquid pharmaceutical formulation including lipid nanoparticles encapsulating mRNA, comprising the sequential steps of:
spray freezing the liquid pharmaceutical formulation, in a column with temperature-controlled walls, the temperature of which is controlled by means of a cooling agent maintained at temperatures between −100 and −190° C., so as to obtain frozen pellets;
transferring the frozen pellets to a vacuum drying chamber; and
drying the frozen pellets within a chamber at a pressure no higher than 1000 microbar, the pellets being heated at a controlled temperature within the chamber.

15-25. (canceled)

26. Lyophilized pharmaceutical product obtained by a process according to claim 14.

27. Product according to claim 26, wherein the Z-average of the LNPs is in the range of 20 to 180 nm, more preferably in the range of 30 to 150 nm, and most preferably in the range of 40 to 120 nm.

28. Product according to claim 26, wherein the PDI of the LNPs is in the range of 0.01 to 0.5, more preferably in the range of 0.05 to 0.4 and most preferably in the range of 0.1 to 0.3.

29. Product according to claim 26, wherein the efficiency of encapsulation is greater than 70%, more preferably greater than 80%, and most preferably greater than 90%.

30. Product according to claim 26, wherein the mRNA has an integrity of at least 50%, preferably of at least 60%, more preferably of at least 70%, more preferably of at least 80%, most preferably of at least 90%.

31. Product according to claim 26, wherein the residual water content is below 4%, more preferably below 2%, and most preferably below 1% about 2 weeks to about 1 month, 2 months, 3 months, 4 months, 5 months, 6 months, 9 months, 1 year, or 2 years after storage at temperatures less than or equal to refrigerated storage.

32-36. (canceled)

37. Product according to claim 26, wherein the mRNA has an in-vitro expression of at least 30%, preferably of at least about 40%, more preferably of at least about 50%, more preferably of at least about 60%, more preferably of at least about 70%, and most preferably of at least about 80% about 2 weeks to about 1 month, 2 months, 3 months, 4 months, 5 months, 6 months, 9 months, 1 year, or 2 years after storage at temperatures less than or equal to refrigerated storage.

38. Product according to claim 26, wherein the sucrose concentration is in the range of 5 to 60%, more preferably in the range of 5 to 25%, and most preferably in the range of 10 to 20%.

39. (canceled)
